(19) United States
(12) Patent Application Publication
(10) Pub. No.: US 2015/0250911 A1
(43) Pub. Date: Sep. 10, 2015

(54) MALODOR COUNTERACTING COMPOSITIONS

(71) Applicant: GIVAUDAN, S.A., Vernier (CH)

(72) Inventors: Stephan Bieri, Oschwand (CH); Thierry Granier, Duebendorf (CH)

(73) Assignee: GIVAUDAN, S.A., Vernier (CH)

(21) Appl. No.: 14/434,009

(22) PCT Filed: Oct. 22, 2013
(86) PCT No.: PCT/EP2013/072065

§ 371 Date: Apr. 7, 2015

(30) Foreign Application Priority Data
Oct. 22, 2012 (GB) 1218904.9

(51) Int. Cl. A61L 9/14; C07C 31/133; C07C 31/135

(52) U.S. Cl. CPC A61L 9/14; C07C 31/1355; C07C 31/133; C07C 2101/14; C07C 2101/20; C07C 2101/18

(57) ABSTRACT

The use of alcohols of formula (I)×

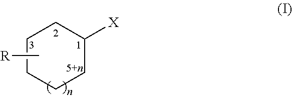

wherein
X is selected from hydroxyl and C₁-C₆ hydroxyalkyl;
I) n is an integer from 6 to 10, and R is selected from hydrogen and methyl; or
II) n is 0 or 1, and R is selected from branched C₃-C₆ alkyl and C₅-C₆ cycloalkyl;
with the proviso that the compound of formula (I) contains 11 to 15 carbon atoms; for the reduction of the sensory perception of malodour.

The present invention refers to malodour reducing compounds. More particularly, the present invention refers to the use of certain alcohols for the reduction of the sensory perception of malodour.

Malodours are offensive, unpleasant odours emitted into an atmosphere from a source of malodour. Typical sources include fabrics, hard surfaces, skin, and hair. Malodours have either personal or environmental origin. For example sweat, urine and feces malodours are personal in origin, whereas kitchen and cooking malodours as well as chemical compounds (organic and inorganic chemical compounds) are of environmental origin.

Amines, thiols, sulfides, short chain aliphatic and olefinic acids, e.g. fatty acids, are typical of the chemicals found in and contributed to sweat, household, and environmental malodours. These types of malodours typically include indole, skatole, and methanethiol found in toilet and animal odours; piperidine and morpholine found in urine; pyridine and triethyl amine found in kitchen and garbage odours; and short chain fatty acids, such as 3-methyl-3-hydroxyhexanoic acid, 3-methylhexanoic acid or 3-methyl-2-hexenoic acid, found in axilla malodours.

Malodours chemical compounds also found in industry, in particular chemical industry, for example, in production sites. The malodour may be originated from compounds indispensable as starting material, or formed as intermediates in a chemical process. For example organic sulfides such as DMDS (dimethyl disulfide) are useful additives for steam cracking, but unfortunately do posses a very unpleasant odour which makes its use difficult. Further, non limiting examples of chemical compounds possessing an unpleasant odour are methyl mercaptan, iso-valeric acid (3-methylbutanoic acid), dimethyl trisulfide, and dimethyl sulfide. Whereas these highly malodorous chemical compounds are essential in some areas, they are unpleasant to handle due to their offensive malodour even when emitted in very low concentrations into an atmosphere.

Several approaches have been used to counteract malodours. These approaches include masking by superimposing the malodour with a pleasant stronger odor, suppression of the malodour by mixing with an ingredient that causes a negative deviation according to Raoult's law, elimination of the malodour by absorption of the malodour by a porous or cage-like structure, and avoidance of the formation of malodours by such routes as antimicrobials and enzyme inhibitors. Although the methods known in the art have the ability to neutralize certain malodours, there still remains a need for further compounds which are even more efficient against malodours.

Surprisingly, inventors found that malodours (strong, unpleasant odour), such as isovaleric acid (IVA), dimethyl disulfide (DMDS), dimethyl trisulfide, 2-methyl-3-mercaptobutan-1-ol, and 3-hydroxy-3-methyl hexanoic acid (HMHA), are not or less noticeable by human when a compound of formula (I) as defined herein below is also present in the atmosphere.

Further, non-limiting examples of offensive malodours are trimethylamine and 1-octen-3-ol.

In a first embodiment there is provided a method of reducing the perception of malodour comprising the step of releasing into an atmosphere containing malodour a compound of formula (I)

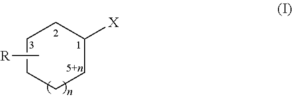

wherein
X is selected from hydroxyl and C₁-C₆ hydroxyalkyl; and
   I) n is an integer from 6 to 10, and R is selected from hydrogen and methyl; or
   II) n is 0 or 1, and R is selected from branched C₃-C₆ alkyl and C₅-C₆ cycloalkyl;
and the compound of formula (I) contains 11, 12, 13, 14 or 15 carbon atoms.

In a further embodiment there is provided a method of reducing the perception of malodour comprising the step of releasing into the atmosphere containing malodour a compound of formula (I)

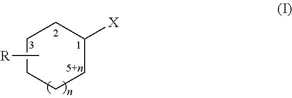

wherein
X is selected from hydroxyl and C₁-C₆ hydroxyalkyl; and
   I) n is an integer from 6 to 10, and R is selected from hydrogen and methyl; or
   II) n is 0 or 1, and R is selected from branched C₃-C₆ alkyl and C₅-C₆ cycloalkyl, with the proviso that for n=0 and X=hydroxyl, R is not attached to C-1; and for n=1 and X=hydroxyl, R is not in alpha- or beta-position to C-1;
and the compound of formula (I) contains 11, 12, 13, 14 or 15 carbon atoms.

The compounds of formula (I) wherein X is hydroxyl, n is an integer from 6 to 10 (e.g. 7, 8 or 9) and R is hydrogen represent particular aspects of the invention.

Another aspect of the invention refers to compounds of formula (I) wherein X is hydroxyl, n is 1, and R is selected from cyclohexyl or C₅ branched alkyl (e.g. 2-methyl but-2-yl).

In a further embodiment compounds of formula (I) are selected from compounds of formula (I) wherein n is 0 or 1, R is C₃-C₄ branched alkyl (e.g. iso-propyl, iso-butyl), and X is selected from C₄-C₅ hydroxyalkyl (e.g. 2-methylpropyl-3-ol, 1,2-dimethylpropyl-3-ol, and 2-butyl-4-ol).

As used in relation to compounds of formula (I), unless otherwise indicated, “hydroxyalkyl” refers to linear and branched C₁-C₆ hydroxyalkyl, preferably to C₃, C₄, C₅ hydroxyalkyl, e.g. 2-methylpropyl-3-ol, 1,2-dimethylpropyl-3-ol, and 2-butyl-4-ol.

Specific examples of compounds of formula (I) may be selected from:
cycloundecanol,
cyclododecanol,
cyclotridecanol,
1-methylcyclotridecanol,
cyclopentadecanol,
4-(tert-pentyl)cyclohexanol,
4-cyclohexylcyclohexanol,
2-cyclohexylcyclohexanol,
3-(4-isobutylcyclohexyl)-2-methylpropan-1-ol,
3-(3-tert-butylcyclohexyl)-2-methylpropan-1-ol,
3-(3-isopropylcyclohexyl)-2-methylbutan-1-ol,
3-(4-isopropylcyclohexyl)-2-methylpropan-1-ol, and
3-(3-isopropylcyclohexyl)-butan-1-ol.

Any means capable of releasing a volatile substance into the atmosphere may be used. In the context of the present invention, the substantive “means” includes any type of air-freshener devices which may include a heater and/or fan and/or nebulization systems well known to the person skilled in the art. Further examples are wick type air-freshener devices.

The compounds of formula (I) may either be used as such or may be diluted with an essentially odourless solvent, such as dipropylene glycol (DPG), isopropyl myristate (IPM), triethyl citrate (TEC), diethylphtalat (DEP) and alcohol (e.g. ethanol).

In a further embodiment the compound of formula (I) as hereinabove defined may be combination with a fragrance. The fragrance may be selected from
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil or ylang-ylang oil;
alcohols, e.g. cinnamic alcohol, cis-3-hexenol, citronellol, Ebanol™, eugenol, farnesol, geraniol, Super Muguet™, linalool, menthol, nerol, phenylethyl alcohol, rhodinol, Sandalore™, terpineol or Timberol™;
aldehydes and ketones, e.g. Azurone® [7-(3-methylbutyl)-1,5-benzodioxepin-3-one], anisaldehyde, α-amylcinnamaldehyde, Georgywood™, hydroxycitronellal, Iso E® Super, Isoraldeine®, Hedione®, Lilial®, maltol, methyl cedryl ketone, methylionone, verbenone, or vanillin;
ether and acetals, e.g. Ambrox®, geranyl methyl ether, rose oxide, or Spirambrene;
esters and lactones, e.g. benzyl acetate, cedryl acetate, α-decalactone, Helvetolide®, γ-undecalactone or vetivenyl acetate;
macrocycles, e.g. Ambrettolide, ethylene brassylate or Exaltolide®; and
heterocycles, e.g. isobutylchinoline.

Extensive sensory studies have revealed that already small concentrations in the atmosphere of compound of formula (I), or a mixture thereof, are efficient enough to reduce or eliminate the sensory perception of malodor also present in the air.

For example, good results have been achieved with 1 to 10 mol 10⁻¹²/l air of a compound of formula (I), or a mixture thereof, e.g. 3 mol×10⁻¹²/l air.

In a further embodiment there is provided a method of reducing the perception of malodour comprising the step of releasing into an atmosphere containing malodour a compound of formula (I) as defined hereinabove in such a concentration that the ratio between the compound of formula (I), or a mixture thereof (malodour blocker), and malodour is from about 1:1 (mol weight) to about 1:100 (mol weight), e.g. between 1:10 to 1:70 (such as 1:30 or 1:50), in the atmosphere.

Whereas some compounds falling within the definition of formula (I) have been described in the literature, there is no indication that they have the ability to reduce the perception of malodour by humans, and thus constitute a further aspect of the present invention.

Accordingly, the present invention refers in a further embodiment to the non-therapeutic use of a compound of formula (I), or a mixture thereof for the reduction of the sensory perception of malodour by a human.

It is believed, without being bond by theory, that the compounds of formula (I) as hereinabove defined do block the cyclic nucleotide-gate ion channel and thus reducing the number of calcium ions that enter the cell and thereby decreasing the activation of the olfactory sensory neurons.

The invention is now further described with reference to the following non-limiting examples. These examples are for the purpose of illustration only, and it is understood that variations and modifications can be made by one skilled in the art.

### EXAMPLE 1

### 3-(4-isobutylcyclohexyl)-2-methylpropan-1-ol

A mixture of 3-(4-isobutylphenyl)-2-methylpropanal (also known under the name Silvial, 80 g, 0.392 mol) and 5% ruthenium on aluminium oxide (4.0 g) was stirred under hydrogen (120 bar) at 130° C. for 72 h. The resulting mixture was dissolved in MTBE (250 ml), filtered and concentrated. The crude oil (81.5 g, 29:71 mixture of diastereomers) was distilled (87-92° C. head temperature, 115-130° C. bath temperature, 0.07-0.06 mbar, fractions 5-10) using a 15 cm-Vigreux column giving olfactorily and chemically pure 3-(4-isobutylcyclohexyl)-2-methylpropan-1-ol (56.6 g, 68%, 25:75 mixture of diastereomers). Some head and tail fractions were joined (13.1 g; fractions 2-4: 84-88° C. head temperature, 110-115° C. bath temperature, 0.07 mbar and fraction 11: 84-92° C. head temperature, 130-150° C. bath temperature, 0.06 mbar) and distilled using a Kugelrohr apparatus (110-120° C., 0.07 mbar) leading to olfactorily and chemically pure 3-(4-isobutylcyclohexyl)-2-methylpropan-1-ol (10.7 g, 13%, 30:70 mixture of diastereomers).

¹H-NMR (CDCl₃, 400 MHz): 25:75 mixture of diastereomers δ 3.50 (dd, J=5.6, 10.6, 0.75H), 3.48 (dd, J=5.4, 10.6, 0.25H), 3.39 (dd, J=6.8, 10.6, 0.75H), 3.37 (dd, J=6.7, 10.6, 0.25H), 1.78-0.94 (m, 17H), 0.91 (d, J=6.8, 2.25H), 0.90 (d, J=6.6, 0.75H), 0.85 (d, J=6.6, 2.25H), 0.84 (d, J=6.6, 0.75H).

¹³C-NMR (CDCl₃, 100 MHz): data of the major isomer δ 68.70 (t), 43.60 (t), 37.78 (t), 33.03 (d), 32.61 (d), 32.42 (d), 29.82 (t), 29.10 (t), 28.92 (t), 28.31 (t), 25.05 (d), 22.85 (q, 2C), 16.80 (q).

data of the minor isomer δ 68.71 (t), 47.04 (t), 41.08 (t), 35.30 (d), 35.02 (d), 34.18 (t), 33.52 (t), 33.44 (t), 32.96 (t), 32.72 (d), 24.77 (d), 22.89 (q), 22.88 (q), 16.85 (q).

MS (EI): data of the major diastereomer 195 (2), 194 (13), 180 (1), 179 (5), 166 (2), 165 (3), 153 (4), 152 (30), 151 (4), 138 (16), 137 (35), 123 (22), 110 (16), 109 (27), 97 (29), 96 (64), 95 (95), 83 (66), 82 (52), 81 (100), 80 (19), 69 (61), 68 (21), 67 (58), 57 (53), 56 (14), 55 (89), 43 (35), 41 (55), 31 (13), 29 (10); data of the minor diastereomer 195 (3), 194 (17), 180 (1), 179 (6), 166 (2), 165 (3), 153 (5), 152 (32), 151 (4), 138 (17), 137 (35), 123 (23), 110 (15), 109 (26), 97 (33), 96 (63), 95 (94), 83 (74), 82 (51), 81 (100), 80 (20), 69 (66), 68 (20), 67 (57), 57 (59), 56 (14), 55 (94), 43 (38), 41 (57), 31 (13), 29 (11).

### EXAMPLE 2

### 3-(3-tert-butylcyclohexyl)-2-methylpropan-1-ol

At 8° C., a mixture of NaBH₄ (1.85 g, 48.9 mmol, 1 eq.) and MeOH (40 ml) was treated dropwise within 10 min. with a solution of 3-(3-tert-butylphenyl)-2-methylpropan-1-al (meta-lysmeral, 10.0 g, 48.9 mmol) in MeOH (60 ml) while the reaction temperature increased to 30° C. The resulting mixture was stirred for 1 h, poured into ice/water (150 ml), treated with a 2M aqueous HCl solution (40 ml), and extracted with MTBE (150 ml). The organic phase was washed with water (100 ml), dried (MgSO₄, 17 g) and concentrated. The crude oil (9.15 g) was distilled using a Kugelrohr apparatus (150° C., 0.1 mbar) giving 3-(3-tert-butylphenyl)-2-methylpropan-1-ol (6.5 g, 64%).

¹H-NMR (CDCl₃, 400 MHz): δ 7.24-7.20 (m, 2H), 7.19-7.17 (m, 1H), 7.01-6.95 (m, 1H), 3.53 (dd, J=5.9, 10.5, 1H), 3.46 (dd, J=6.3, 10.6, 1H), 2.74 (dd, J=6.3, 13.4, 1H), 2.41 (dd, J=7.9, 13.5, 1H), 2.01-1.87 (m, 1H), 1.72 (br. s, OH), 1.31 (s, 9H), 0.92 (d, J=6.6, 3H).

¹³C-NMR (CDCl₃, 100 MHz): δ 151.09 (s), 140.19 (s), 127.92 (d), 126.22 (d), 126.20 (d), 122.77 (d), 67.77 (t), 40.05 (t), 37.86 (d), 34.55 (s), 31.40 (q, 3C), 16.54 (q).

A mixture of a solution of 3-(3-tert-butylphenyl)-2-methylpropan-1-ol (6.5 g, 31.5 mmol) in MeOH (30 ml) and 5% ruthenium on aluminium oxide (1.0 g) was stirred under hydrogen (180 bar) at 140° C. for 1.5 h. The resulting mixture was filtered and concentrated giving crude 3-(3-tert-butylcyclohexyl)-2-methylpropan-1-ol (6.7 g, quantitative, 38:33:15:14 diastereomeric mixture).

¹H-NMR (CDCl₃, 400 MHz): δ 3.74-3.34 (m, 2H), 2.01-0.46 (m, 14H), 0.94-0.89 (4d, J=6.3-6.6, 3H), 0.84-0.82 (2s, 9H).

¹³C-NMR (CDCl₃, 100 MHz): selected signals δ 68.84 (t), 68.70 (t), 68.67 (t), 68.60 (t), 48.02 (d), 47.93 (d), 41.52 (d), 41.50 (t), 41.30 (d), 35.39 (t), 35.60 (d), 35.26 (d), 34.91 (t), 34.75 (t), 34.13 (t), 34.11 (t), 33.59 (d), 33.52 (d), 32.85 (t), 32.65 (d), 32.58 (d), 32.45 (s), 32.41 (s), 32.27 (s), 32.22 (t), 30.95 (t), 30.34 (t), 30.29 (d), 30.23 (d), 29.17 (t), 27.67 (t), 27.53 (q), 27.39 (q), 26.75 (t), 26.67 (t), 21.62 (t), 21.36 (1), 18.35 (q), 17.01 (q), 16.78 (q), 16.71 (q). MS (EI): data of the major diastereomer 197 (1), 179 (1), 156 (3), 155 (26), 138 (6), 137 (16), 109 (5), 96 (23), 95 (52), 83 (21), 81 (67), 69 (23), 67 (33), 57 (100), 56 (62), 55 (43), 43 (13), 41 (39), 31 (6), 29 (9).

### EXAMPLE 3

### 3-(3-isopropylcyclohexyl)-2-methylbutan-1-ol

To a mixture of 3-(3-isopropylphenyl)butanal (florhydral, 20 g, 0.105 mol), formaldehyde (8.65 g, 37% solution in water, 0.105 mol, 1 eq.), isopropanol (15 ml) was successively added propionic acid (0.78 g, 10.5 mmol, 0.1 eq.) and pyrrolidin (0.75 g, 10.5 mmol, 0.1 eq., added in three fractions) while the reaction temperature increased to 40′. The resulting mixture was stirred at 45° C. for 3 h, cooled, poured into aqueous saturated NaHCO₃ solution (100 ml) and extracted three times with MTBE (100 ml). The combined organic phases were washed with water (100 ml), aqueous saturated NaCl (100 ml), dried (MgSO₄) and concentrated. Short-path distillation (76-79° C. head temperature, 95-100° C. bath temperature, 0.05 mbar) of the crude oil (21.3 g) gave 3-(3-isopropylphenyl)-2-methylenebutanal (17.9 g, 84%).

¹H-NMR (CDCl₃, 400 MHz): δ 9.53 (s, 1H), 7.23-7.18 (m, 1H), 7.08-7.05 (m, 2H), 7.04-7.00 (m, 1H), 6.21 (d, J=1.0, 1H), 6.05 (s, 1H), 4.02 (q, J=7.1, 1H), 2.87 (hept, J=6.9, 1H), 1.43 (d, J=7.3, 3H), 1.23 (d, J=6.8, 6H).

¹³C-NMR (CDCl₃, 100 MHz): δ 193.85 (s), 154.58 (s), 148.99 (s), 143.53 (s), 133.55 (t), 128.33 (d), 125.94 (d), 124.81 (d), 124.39 (d), 37.26 (d), 34.10 (d), 24.05 (q), 23.98 (q), 20.05 (q). MS (EI): 202 (7), 187 (4), 169 (2), 160 (13), 159 (100), 145 (7), 141 (6), 131 (18), 129 (9), 128 (11), 117 (10), 115 (15), 105 (11), 91 (20), 77 (8), 55 (5), 43 (10).

A mixture of 3-(3-isopropylphenyl)-2-methylenebutanal (10 g, 49 mmol) and 5% ruthenium on aluminium oxide (1.0 g) was stirred under hydrogen (110 bar) at 130° C. for 22 h. The resulting mixture was dissolved in MTBE (50 ml), filtered and concentrated. The crude oil (9.3 g, 18:26:22:21:4:4 mixture) was distilled (82-870° C. head temperature, 135-160° C. bath temperature, 0.05 mbar, fractions 3-6) using a 7.5 cm-Vigreux column giving olfactorily and chemically pure 3-(3-isopropylcyclohexyl)-2-methylbutan-1-ol (4.25 g, 40%, 19:28:23:22:4:4 diastereomeric mixture).

¹H-NMR (CDCl₃, 400 MHz): δ 3.73-3.33 (m, 2H), 1.91-0.56 (m, 26H).

¹³C-NMR (CDCl₃, 100 MHz): selected signals δ7.53 (t), 67.49 (t), 67.30 (t), 67.25 (t), 66.13 (t), 66.04 (t), 44.42 (d), 44.24 (d), 44.20 (d), 44.15 (d), 40.75 (d), 40.73 (d), 40.65 (d), 40.52 (d), 39.69 (d), 39.55 (d), 38.77 (d), 38.66 (d), 37.91 (d), 37.70 (d), 36.63 (d), 36.56 (d), 35.73 (t), 34.93 (t), 33.46 (t), 33.14 (d), 33.11 (d), 33.10 (d), 32.12 (t), 31.46 (t), 31.39 (t), 29.86 (t), 29.68 (t), 29.61 (t), 29.49 (t), 29.45 (t), 29.29 (t), 27.81 (t), 26.80 (t), 26.72 (t), 26.65 (t), 26.59 (t), 20.60 (q), 19.89 (q), 19.88 (q), 19.61 (q), 19.57 (q), 19.51 (q), 16.03 (q), 15.94 (q), 12.39 (q), 12.37 (q), 11.92 (q), 11.80 (q), 11.36 (q), 11.32 (q). MS (EI): data of the major diastereomer 194 (1), 179 (1), 165 (2), 152 (23), 151 (17), 137 (4), 125 (20), 124 (11), 123 (8), 111 (10), 110 (10), 109 (25), 97 (29), 95 (24), 83 (53), 82 (38), 81 (28), 70 (36), 69 (100), 67 (29), 57 (33), 55 (65), 43 (29), 41 (51).

### EXAMPLE 4

### 3-(3-isopropylcyclohexyl)-butan-1-ol

A mixture of 3-(3-isopropylphenyl)butanal (florhydral, 10 g, 0.052 mol) and 5% ruthenium on aluminium oxide (0.5 g) was stirred under hydrogen (60 bar) at 130° C. for 10 h. The resulting mixture was filtered over celite (rinsed with cyclohexane). Concentration followed by distillation of the crude oil (10.6 g, 1:1 mixture) using a Kugelrohr apparatus (160° C., 11 mbar) gave olfactorily and chemically pure 3-(3-isopropylcyclohexyl)-3-butan-1-ol (7.8 g, 75%, 1:1 diastereomeric mixture).

¹H-NMR (CDCl₃, 400 MHz): δ 3.73-3.55 (m, 2H), 2.51 (s, OH), 1.83-1.74 (m, 1H), 1.71-1.53 (m, 4H), 1.51-1.30 (m, 3H), 1.29-1.13 (m, 2H), 1.12-1.01 (m, 1H), 1.01-0.66 (m, 12H).

¹³C-NMR (CDCl₃, 100 MHz): δ 61.38 (t), 61.35 (t), 44.31 (d), 44.20 (d), 43.00 (d), 42.86 (d), 37.19 (t), 36.86 (t), 34.62 (d), 34.61 (d), 33.93 (t), 33.11 (d), 33.08 (d), 32.09 (t), 30.20 (t), 29.73 (t), 29.67 (t), 28.30 (t), 26.68 (t), 26.59 (t), 19.85 (q), 19.82 (q), 19.64 (q), 19.59 (q), 16.21 (q), 15.95 (q). MS (EI): 180 (1), 152 (28), 137 (26), 125 (33), 124 (18), 123 (9), 110 (6), 109 (26), 96 (10), 95 (34), 83 (46), 82 (49), 81 (52), 69 (100), 67 (35), 57 (30), 55 (54), 43 (21), 41 (34).

### EXAMPLE 5

### Sensory Studies

Sensory studies were performed in 120 ml Beetson jar. The test compounds were prepared in DEP (=Diethylphtalate) and pipetted onto a desized woven cotton piece of cloth placed at the bottom of a jar. Where two or more compounds were tested in the same jar, they were applied to separate areas of the cotton cloth. Prior the first assessment and between assessments, the jars were incubated at room temperature for one hour so that the headspace did reach equilibrium.

For the sensory test, participants were given a reference malodour sample (comprising the same ingredients as the jar with the malodour) and were told that this scored 4 on a 0-5 scale. They were then asked to assess and score three test jars on a 0-5 scale in the specified sequence, with a 20 second gap between jars. The jars were given to the assessors blind in random permutations, with each volunteer smelling all three permutations in duplicate.

### Score Scale

0: no malodour present
1: strong reduction in malodour
2 medium reduction in malodour
3: weak malodour reduction
4: no change from control
5: worse than control

The three test jars contained:
DEP, a neutral non-volatile solvent, used as a hidden positive control,
the malodour used as a hidden negative control in a concentration as indicated in Table 1 below, and
the malodour plus the test compound (compound of formula (I)) in concentrations as indicated in Table 1 below.

Approx. 20 trained volunteers did participate in each study twice. The results are shown in Table 1 below.

TABLE 1

| Jar | | Malodour Score* | STD Error |
|---|---|---|---|
| a-1 | DMDS @ 0.06% | 3.72 | 0.085 |
| b-1 | DMDS (50 ul @ 0.06%) | 0.91 | 0.123 |
| | and 200 ul ORIVOL | | |
| c-1 | DEP | 0.11 | 0.040 |
| a-2 | DMDS @ 0.5% | 3.51 | 0.121 |
| b-2 | DMDS (50 ul @ 0.5%) | 1.46 | 0.156 |
| | and 200 ul ORIVOL | | |
| c-2 | DEP | 0.14 | 0.044 |
| a-3 | DMDS @ 0.5% | 3.489 | 0.067 |
| b-3 | DMDS 50 ul@ 0.5% and 200 ul | 2.862 | 0.137 |
| | ORIVOL @0.1% | | |
| c-3 | DEP | 0.283 | 0.063 |

*arith. means
DMDS = dimethyl disulfide
DEP = diethylphtalate
ORIVOL = 4-(tert-pentyl)cyclohexanol

As can be seen from the results in Table 1 above, ORIVOL was found to provide a significant reduction on malodour perceivable by the human nose compared to the malodour taken alone, even at very low concentrations.

### EXAMPLE 6

### Sensory Studies

Sensory studies were performed following the procedure described in Example 5. For the sensory test, participants were given a reference malodour sample (comprising the same ingredients as the jars with the maldodour) and were told that this scored 4 on a 0-5 scale. They were then asked to assess and score three test jars on a 0-5 scale in the specified sequence, with a 20 second gap between jars. The jars were given to the assessors blind in random permutations, with each volunteer smelling all three permutations in duplicate.

The three test jars contained:
two jars containing the malodour used as a hidden negative control in a concentration as indicated in Table 2 below, and
one jar containing the malodour plus the test compound (compound of formula (I)) in concentrations as indicated in Table 2 below.

### Score Scale

0: no malodour present
1: strong reduction in malodour
2 medium reduction in malodour
3: weak malodour reduction
4: no change from control
5: worse than control

Approx. 20 trained volunteers did participate in each study twice. The results are shown in Table 2 below.

TABLE 2

| Jar | | Malodour Score* | STD Error |
|---|---|---|---|
| a-4 | MO (50 ul @ 0.005%) | 3.195 | 0.136 |
| b-4 | MO (50 ul @ 0.005%) and 200 ul | 2.375 | 0.177 |
| | ORIVOL @ 0.1% | | |
| c-4 | MO (50 ul @ 0.005%) | 2.945 | 0.132 |
| a-5 | IVA (50 ul @ 0.06%) | 3.194 | 0.122 |
| b-5 | IVA (50 ul @ 0.06%) and 200 ul | 2.733 | 0.140 |
| | ORIVOL @0.1% | | |
| c-5 | IVA (50 ul @ 0.06%) | 3.049 | 0.129 |
| a-6 | HMHA (50 ul @ 0.01%) | 3.222 | 0.348 |
| b-6 | HMHA (50 ul @ 0.01%) and | 2.194 | 0.388 |
| | 200 ul ORIVOL @ 0.1% | | |
| c-6 | HMHA (50 ul @ 0.01%) | 2.500 | 0.369 |

*arith. means
ORIVOL = 4-(tert-pentyl)cyclohexanol
MO = 2-methyl-3-mercaptobutan-1-ol
IVA = isovaleric acid
HMHA = 3-hydroxy-3-methylhexanoic acid

As can be seen from the results in Table 2 above, ORIVOL was found to provide a significant reduction on malodour perceivable by the human nose compared to the malodour taken alone.

### EXAMPLE 7

### Sensory Tests

The same procedures as described in Example 5 have been followed.

The compounds of formula (I) were assessed by the trained volunteers against 4 malodour compounds, namely isovaleric acid, dimethyl disulfide, 2-methyl-3-mercaptobutan-1-ol, and 3-hydroxy-3-methyl hexanoic acid.

For the sensory test, participants were given the reference malodour sample and were told that this scored+on a scale as indicated below. They were then asked to assess and score the test jars in the specified sequence, with a 20 second gap between jars. The jars were given to the assessors blind in random permutations, with each volunteer smelling all permutations in duplicate. The results are given in Table 3 below.

The following score scale was used:

++++: no malodour present/strong reduction in malodour
+++: medium reduction in malodour
++: weak malodour reduction
+: no change from control
◯: worse than control

TABLE 3

| | | Sensory |
|---|---|---|
| Compound | | score |
| 5.1 | 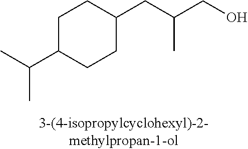 | ++++ |
| 5.2 (Ex. 3) | 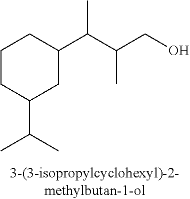 | ++++ |
| 5.3 (Ex. 2) | 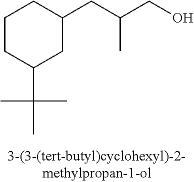 | ++++ |
| 5.4 | 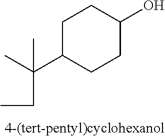 | ++++ |
| 5.5 | 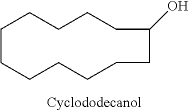 [SMILES: OC1CCCCCCCCCCC1] | +++ |
| 5.6 (Ex. 1) | 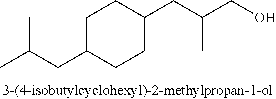 | +++ |
| 5.7 | 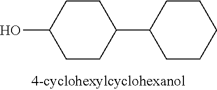 [SMILES: OC1CCC(C2CCCCC2)CC1] | +++ |
| 5.8 (Ex. 4) | 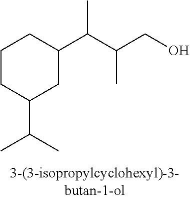 | ++++ |
| 5.9 | 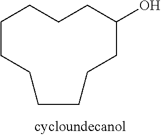 [SMILES: OC1CCCCCCCCCC1] | +++ |
| 5.10 | 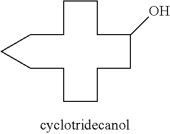 [SMILES: OC1CCCCCCCCCCCC1] | ++++ |
| 5.11 | 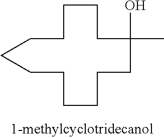 | ++++ |
| 5.12 | 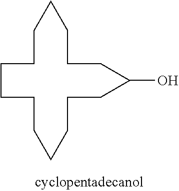 [SMILES: OC1CCCCCCCCCCCCCC1] | ++++ |

As can be seen from Table 3 above, all compounds tested showed good malodour reduction properties against malodour compounds.

1. A method of reducing the perception of malodour comprising the step of releasing into an atmosphere containing malodour a compound of formula (I) 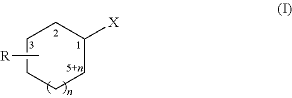
wherein
X is selected from hydroxyl and C₁-C₆ hydroxyalkyl;
I) n is an integer from 6 to 10, and R is selected from hydrogen and methyl; or
II) n is 0 or 1, and R is selected from branched C₃-C₆ alkyl and C₅-C₆ cycloalkyl;
with the proviso that the compound of formula (I) contains 11 to 15 carbon atoms.

2. The method according to claim 1 wherein the compound is selected from compounds of formula (I) 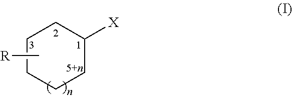
wherein
X is selected from hydroxyl and C₁-C₆ hydroxyalkyl; and
I) n is an integer from 6 to 10, and R is selected from hydrogen and methyl; or
II) n is 0 or 1, and R is selected from branched C₃-C₆ alkyl and C₅-C₆ cycloalkyl, with the proviso that for n=0 and X=hydroxyl, R is not attached to C-1; and for n=1 and X=hydroxyl, R is not in alpha- or beta-position to C-1;
with the proviso that the compound of formula (I) contains 11-15 carbon atoms.

3. The method of claim 2 wherein the compound of formula (I) or a mixture thereof is released in such a concentration that the ratio between the compound of formula (I) and the malodour is from about 1:1 to about 1:100 in the atmosphere.

4. The method according to claim 2 wherein the compound of formula (I) is selected from cycloundecanol, cyclododecanol, cyclotridecanol, 1-methylcyclotridecanol, cyclopentadecanol, 4-(tert-pentyl)cyclohexanol, 4-cyclohexylcyclohexanol, 2-cyclohexylcyclohexanol, 3-(4-isobutylcyclohexyl)-2-methylpropan-1-ol, 3-(3-tert-butylcyclohexyl)-2-methyl propan-1-ol, 3-(3-isopropylcyclohexyl)-2-methylbutan-1-ol, 3-(4-isopropylcyclohexyl)-2-methylpropan-1-ol, and 3-(3-isopropylcyclohexyl)-butan-1-ol.

5. (canceled)

6. An effective amount of a compound of formula (I) 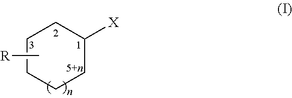
wherein
X is selected from hydroxyl and C₁-C₆ hydroxyalkyl;
I) n is an integer from 6 to 10, and R is selected from hydrogen and methyl; or
II) n is 0 or 1, and R is selected from branched C₃-C₆ alkyl and C₅-C₆ cycloalkyl;
with the proviso that the compound of formula (I) contains 11 to 15 carbon atoms, for use to reduce the sensory perception of malodour.

7. The method of claim 1 wherein the compound of formula (I) or a mixture thereof is released in such a concentration that the ratio between the compound of formula (I) and the malodour is from about 1:1 to about 1:100 in the atmosphere.

8. The method according to claim 7 wherein the compound of formula (I) is selected from cycloundecanol, cyclododecanol, cyclotridecanol, 1-methylcyclotridecanol, cyclopentadecanol, 4-(tert-pentyl)cyclohexanol, 4-cyclohexylcyclohexanol, 2-cyclohexylcyclohexanol, 3-(4-isobutylcyclohexyl)-2-methylpropan-1-ol, 3-(3-tert-butylcyclohexyl)-2-methylpropan-1-ol, 3-(3-isopropylcyclohexyl)-2-methylbutan-1-ol, 3-(4-isopropylcyclohexyl)-2-methylpropan-1-ol, and 3-(3-isopropylcyclohexyl)-butan-1-ol.

9. The method according to claim 1 wherein the compound of formula (I) is selected from cycloundecanol, cyclododecanol, cyclotridecanol, 1-methylcyclotridecanol, cyclopentadecanol, 4-(tert-pentyl)cyclohexanol, 4-cyclohexylcyclohexanol, 2-cyclohexylcyclohexanol, 3-(4-isobutylcyclohexyl)-2-methylpropan-1-ol, 3-(3-tert-butylcyclohexyl)-2-methylpropan-1-ol, 3-(3-isopropylcyclohexyl)-2-methylbutan-1-ol, 3-(4-isopropylcyclohexyl)-2-methylpropan-1-ol, and 3-(3-isopropylcyclohexyl)-butan-1-ol.
